(19) United States
(12) Patent Application Publication
(10) Pub. No.: US 2015/0232558 A1
(43) Pub. Date: Aug. 20, 2015

(54) STIMULATING BONE FORMATION BY INHIBITION OF CD28 CO-STIMULATION

(71) Applicants: EMORY UNIVERSITY, Atlanta, GA (US); UNITED STATES GOVERNMENT REPRESENTED BY THE DEPARTMENT OF VETERANS AFFAIRS, Washington, DC (US)

(72) Inventor: M. Neale Weitzmann, Decatur, GA (US)

(21) Appl. No.: 14/422,600

(22) PCT Filed: Oct. 30, 2013
(86) PCT No.: PCT/US2013/067418

§ 371 Date: Feb. 19, 2015

Related U.S. Application Data
(60) Provisional application No. 61/720,148, filed on Oct. 30, 2012.

(51) Int. Cl. C07K 16/28; C07K 14/705; A61K 39/395; A61K 45/06

(52) U.S. Cl. CPC C07K 16/2827; A61K 45/06; C07K 16/2818; C07K 14/70521; A61K 39/3955; C07K 2319/30

(57) ABSTRACT

The disclosure relates to methods of growing bone or increasing bone mineral density and/or volume comprising administering an effective amount of a therapeutic containing abatacept or belatacept or other molecule that binds with CD8O, CD86, or CD28 providing inhibition of CD28 co-stimulation to a subject in need thereof.

### CROSS-REFERENCE TO RELATED APPLICATIONS

This Application is a 35 U.S.C. 371 of International Application No. PCT/US2013/067418 filed on Oct. 30, 2013, and claims the benefit of priority to U.S. Provisional Application No. 61/720,148 filed on Oct. 30, 2012, which applications are hereby incorporated by reference in their entireties.

### FIELD

The disclosure relates to methods of growing bone or increasing bone mineral density and/or volume comprising administering an effective amount of a therapeutic containing abatacept or belatacept or other molecule that binds with CD80, CD86, or CD28 providing inhibition of CD28 co-stimulation to a subject in need thereof

### BACKGROUND

The standard of care for osteoporosis is to apply anti-resorptive (anticatabolic) therapies once bone has already been lost and osteoporosis has been diagnosed in an attempt to prevent further osteoclastic breakdown. However, because osteoclastic bone breakdown and osteoblastic bone formation are coupled processes, anti-resorptive therapies not only lower bone breakdown (resorption) but simultaneously reduce new bone formation. This makes it difficult to restore bone mass to a normal healthy level. The quality of any new bone thus formed may not be optimal as osteoclasts are needed for normal bone remodeling. Micro-damage thus accumulates with antiresorptive therapy in the long term limiting the duration of use. Teriparatide, also known as anabolic parathyroid hormone (PTH), is a fragment of human PTH that when injected intermittently initiates new bone formation. While parathyroid hormone is effective in many patients, it is undesirable due to the need for daily injections for extended periods, waning efficacy, and the potential for certain cancers such as osteosarcoma. Thus, there is a need for improved therapies to prevent osteoporosis and grow bone.

CD28 is a molecule expressed on T cells that provides co-stimulatory signals needed for T cell activation. CD28 is the receptor for CD80 (B7.1) and CD86 (B7.2). CTLA4 is similar to CD28 in that both molecules bind to CD80 and CD86. However, CTLA4 blocks the stimulatory signal from being transmitted to T cells, whereas CD28 transmits a stimulatory signal.

Abatacept is a CTLA4-Ig chimeric protein consisting of the extracellular binding domain of CTLA-4 linked to the Fc domain of a human IgG. Abatacept is approved for the treatment of rheumatoid arthritis (RA) and in clinical trials for a number of other autoimmune indications. Additional CTLA4-Ig chimeras are provided in U.S. Published Application Number 2011/0287032. CTLA4-Ig is capable of mitigating ovariectomy-induced bone loss in mice by dampening inflammatory cascades and reducing T-cell activation and expression of TNFα by disrupting communication between T-cells and dendritic cells. Grassi et al., Proc Natl Acad Sci USA, 2007, 104(38):15087-15092. CTLA4-Ig was shown to ameliorate bone loss in a murine model of hyperparathyroidism. Bedi et al., Ann NY Acad Sci, 2010, 1192(1):215-221. CTLA4-Ig has also been demonstrated to directly suppress osteoclast differentiation in the absence of T-cells in vitro and to inhibit inflammatory bone erosion in vivo in an animal model of TNFa-induced arthritis. Axmann et al., Ann Rheum Dis, 2008, 67(11):1603-1609.

Wnt genes express cell surface signaling proteins that bind cell surface receptors such as frizzled receptors and low-density lipoprotein receptor-related proteins. Low-density lipoprotein receptor-related protein 5 (LRPS) functions in bone to regulate bone mass. Increasing LRPS signaling in mature bone cells may be a strategy for treating human disorders associated with low bone mass. See Cui et al., Nat Med. 2011, 17(6):684-91.

References cite herein are not an admission of prior art.

### SUMMARY

The disclosure relates to methods of growing bone or increasing bone mineral density and/or volume comprising administering an effective amount of a therapeutic containing abatacept or belatacept or other molecule that binds with CD80, CD86, or CD28 providing inhibition of CD28 co-stimulation to a subject in need thereof In certain embodiments, the disclosure relates to treating or preventing osteoporosis by administering abatacept or belatacept or other molecule that binds with CD80, CD86, or CD28 providing inhibition of CD28 co-stimulation optionally in combination with another therapeutic agent to a subject in need thereof.

In certain embodiments, the molecule is selected from an antibody, antibody fragment, antibody chimera, antibody mimetic, and aptamer. In certain embodiments, the molecule comprises a polypeptide comprising 100 or more amino acids that overlap with a human CTLA-4 (Cytotoxic T-Lymphocyte Antigen 4) extra cellular domain sequence. In certain embodiments, the polypeptide comprises (SEQ ID NO:1) M HVAQ PAVV LASS RGIA SFVC EYAS PGK(X¹) TEVR VTVL RQAD SQVTE VCAA (X²)Y(X³)M GNEL TF(X⁴)D DSIC TGTS SGNQ VNLT IQGL RAMD TGLY IC(X⁵)V ELMY PPPY YLGI GNGT QIYV IDPE PCPDS D wherein X¹ is Y, A, or H, X² is T or N, X³ is M or Y, X⁴ is L or E, and X⁵ is K or Q and a human immunoglobulin such as used in abatacept or belatacept.

In certain embodiments, the subject is diagnosed with, at risk of, or exhibiting symptoms of bone degenerative disease, osteoporosis, osteopenia, bone metastasis, multiple myeloma, osteogenesis imperfecta, hyperparathyroidism, or alcoholism. In certain embodiments, the subject is diagnosed with a bone density that is more than one or two standard deviations below the mean of a thirty year old subject of the same sex. In certain embodiments, the subject is premenopausal woman, male under the age of 50, male or female under the age of 40 or 30. In certain embodiments, the subject is taking or has taken a glucocorticoid therapy. In certain embodiments, the subject is diagnosed with HW and/or on an antiretroviral therapy with osteopenia or osteoporosis.

In certain embodiments, the antiretroviral therapy is (ART/HAART/cART) zidovudine, lamivudine, abacavir, zidovudine, lopinavir, ritonavir, raltegravir, tenofovir, emtricitabine, efavirenz , rilpivirine, elvitegravir, cobicistat, darunavir, atazanavir, prodrugs, salts, or combinations thereof.

In certain embodiments, the polypeptide is administered in combination with teriparatide, anabolic parathyroid hormone or fragment, calcium, vitamin D, strontium ranelate, raloxifene, denosumab, estrogen, testosterone, bisphosphonates such as pamidronate, alendronate, risedronate, medronate, oxidronate, etidronate, clodronate, tiudronate, neridronate, olpadronate, ibandronate, or zoledronate, or statins such as atorvastatin, cerivastatin, fluvastatin, lovastatin, mevastatin, pitavastatin, pravastatin, rosuvastatin, or simavastatin, or a glucocorticoid such as hydrocortisone, cortisone, prednisone, prednisolone, methylprednisolone, dexamethasone, betamethasone, fludrocortisone, deoxycorticosterone, or aldosterone.

In certain embodiments, the polypeptide is administered in combination with cathepsin K suppressor, odanacatib, sclerostin inhibitor, sclerostin antibody, sclerostin mutant, or AMG-785/CDP-7851.

In certain embodiments, the subject is or is not diagnosed with rheumatoid arthritis. In certain embodiments, the subject is diagnosed with a bone fracture or received spinal fusion surgery.

In certain embodiments, the disclosure relates to osteogenic promoting implant or bone graft compositions comprising the polypeptides disclosed herein optionally comprising a bone morphogenetic protein and/or another growth factor. In certain embodiments, the bone morphogenetic protein is BMP-2, BMP-5, or BMP-7. In certain embodiments, the implant further comprises calcium phosphates and/or bone granules, hydroxyapatite and/or beta-tricalcium phosphate, alpha-tricalcium phosphate, polysaccharides or combinations thereof. In certain embodiments, the implant further comprises crushed bone granules, a hydrogel matrix, or collagen matrix.

In certain embodiments, the disclosure relates to methods of growing bone comprising inserting an osteogenic promoting implant in an area of a subject of desired bone growth and administering an effective amount of a molecule that binds with CD80, CD86, or CD28 providing inhibition of CD28 co-stimulation to the subject.

### BRIEF DESCRIPTION OF THE FIGURES

FIG. 1A shows data indicating CTLA4-Ig promotes accretion of BMD and bone mass in young and skeletally mature mice. Total body BMD (Total) and BMD at lumbar spine (Spine), femur and tibia were quantified by DEXA in young mice at 3 months

FIG. 1B is at, 6 months, after administration of Ig (control) or CTLA4-Ig.

FIG. 1C shows skeletally mature mice 6 months after CTLA4-Ig administration. Graphical data are presented as mean±SEM. Δ=percentage change from Ig. *P<0.05; **P<0.01, ***P<0.001. p=n.s. (not significant). Young Mice 3 months treatment n=10 mice/group; Young mice treated for 6 months, n=11 and 12 for Ig and CTLA4-Ig respectively. Mature mice n=15 mice/group. Representative high-resolution (6 μm) μCT reconstructions of vertebral cancellous bone for CTLA4-Ig or Ig are presented below the DEXA for each group. Scale Bar=500 μm.

FIG. 2A shows data indicating CTLA4-Ig promotes bone formation in mice. Serum markers of bone formation (osteocalcin) in young and skeletally mature mice treated with CTLA4-Ig or Ig for 3 and/or 6 months. Graphical data are presented as mean±SD. ***P<0.001.

FIG. 2B is- representative of calcein double fluorescent labels in femurs from young mice treated for 6 months with CTLA4-Ig or Ig, used to compute mineral apposition and bone formation rates (40× mag.).

FIG. 2C, is representative of the histological sections of Goldner's trichrome stained distal femur in young mice treated for 6 months with CTLA4-Ig or Ig. Mineralized bone stains green. Trabecular bone in bone marrow cavity indicated by yellow arrows and epiphyseal bone by red arrows. (100× mag).

FIG. 3A shows data the bone anabolic Wnt ligand, WntlOb is potently upregulated by CTLA4-Ig in vivo and suppressed by CD28 activation in vitro. Wnt10b production in Ig and CTLA4-Ig treated mice was quantified in whole bone marrow by real time RT-PCR and in conditioned media from purified T cells by ELISA. N=4 mice/group, Mean±SEM. *P<0.05.

FIG. 3B shows WntlOb expression quantified by RT-PCR in purified T cells activated by CD3 antibody with and without CD28 activating antibody. Mean±SD of 12 individual wells/group and representative of 2 independent experiments.

FIG. 3C shows WntlOb quantified by RT-PCR in T cells activated in vitro by antigen presenting cells (APC) +/−CTLA4-Ig. Mean±SD of 3 individual wells/group and representative of 2 independent experiments. ***P<0.001.

FIG. 3D is a Model for anabolic response of CTLA4-Ig involving Wnt10b expression by T cells.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of immunology, medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

In this specification and in the claims that follow, reference will be made to a number of terms that shall be defined to have the following meanings unless a contrary intention is apparent.

Prior to describing the various embodiments, the following definitions are provided and should be used unless otherwise indicated.

As used herein, the term “combination with” when used to describe administration with an additional treatment means that the agent may be administered prior to, together with, or after the additional treatment, or a combination thereof.

As used herein, “subject” refers to any animal, typically a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

### Stimulation of Bone Formation by Preventing T Cell Activation

The disclosure relates to methods of growing bone or increasing bone mineral density and/or volume comprising administering an effective amount of a therapeutic containing abatacept or belatacept or other molecule that binds with CD80, CD86, or CD28 providing inhibition of CD28 co-stimulation to a subject in need thereof.

Because CTLA4-Ig disrupts co-stimulatory interactions between B-cells and T-cells, it has the potential to not only lower immune activation responsible for driving inflammation, but also to disrupt basal bone turnover by disturbing the immuno-skeletal interface and B-cell OPG production. This effect has the potential to offset the gains in bone mass associated with reduced inflammation.

The net effect of CTLA4-Ig on basal bone turnover and mass in mice was investigated by quantifying indices of bone structure and turnover. CTLA4-Ig led to significant bone accrual but surprisingly as a consequence of increased bone formation, as a likely consequence of T-cell expression of the bone anabolic ligand Wnt10b. It has been discovered that CTLA4-Ig leads to induction of bone formation and may have potential applications as a novel bone anabolic agent.

Experiments disclosed herein indicate that pharmacological suppression of CD28 costimulation by CTLA4-Ig results in a bone anabolic signal, a likely consequence of T-cell Wnt10b secretion.

Although the capacity of the immune system to regulate bone resorption though perturbations of the immunoskeletal interface is reported, little is known regarding the ability of the immune system to regulate bone formation. Activated T-cells have been reported to secrete cocktails of cytokines that cumulatively have the capacity to induce alkaline phosphatase activity in purified human bone marrow stromal cells and elevate expression of Runx2 and osteocalcin mRNA. By contrast a number of cytokines involved in immune regulation such as IL-7 or produced by immune cells including T-cells and macrophages, such as TNFα, may act to uncouple bone formation from resorption under inflammatory conditions. Thus, by suppressing the natural compensatory increase in bone formation in response to increased bone resorption bone homeostasis is disrupted leading to bone loss.

CTLA4-Ig has been reported to ameliorate short-term ovariectomy-induced bone loss by reducing T-cell activation and inflammatory cascades and to directly suppress osteoclast differentiation in vitro and inflammatory bone erosion in vivo in an animal model of TNFα-induced arthritis.

A model is proposed whereby WntlOb production is an unintended consequence of abortive T-cell activation due to disruption of the dual-signal mechanism of T-cell activation. Two signals are required for full T-cell activation. The first signal is generated when the TCR engages MHC class I or II bearing antigens on the surface of professional APC (B-cells, macrophages and dendritic cells). This first signal is insufficient for T-cell activation and on its own simply renders T-cells unresponsive to further antigenic stimuli (anergy). At the molecular level this signal involves activation of the cAMP second messenger system. The generation of cAMP leads to activation of protein kinase A (PKA) and cAMP response element (CRE) binding (CREB) protein, a transcription factor that transactivates genes involved in T-cell regulation and anergy. This costimulatory signal involves binding of the CD28 receptor on T-cells, with B7 molecules (B7-1 (CD80) and B7-2 (CD86)), expressed on professional APC (including B-cells, macrophages and dendritic cells). Activation of CD28 leads to induction of phosphodiesterase, an enzyme that cleaves cAMP, neutralizing its second messenger activity and eliminating the repressive first signal, thus preventing anergy and allowing full T-cell activation to proceed. These two signals lead to full T-cell activation, cytokine production, clonal expansion, and prevention of anergy. In the context of normal bona fidae immunological actions cAMP generation would be short lived as the “verification signal” transduced though CD28 would rapidly shut off this pathway, preventing release of Wnt10b.

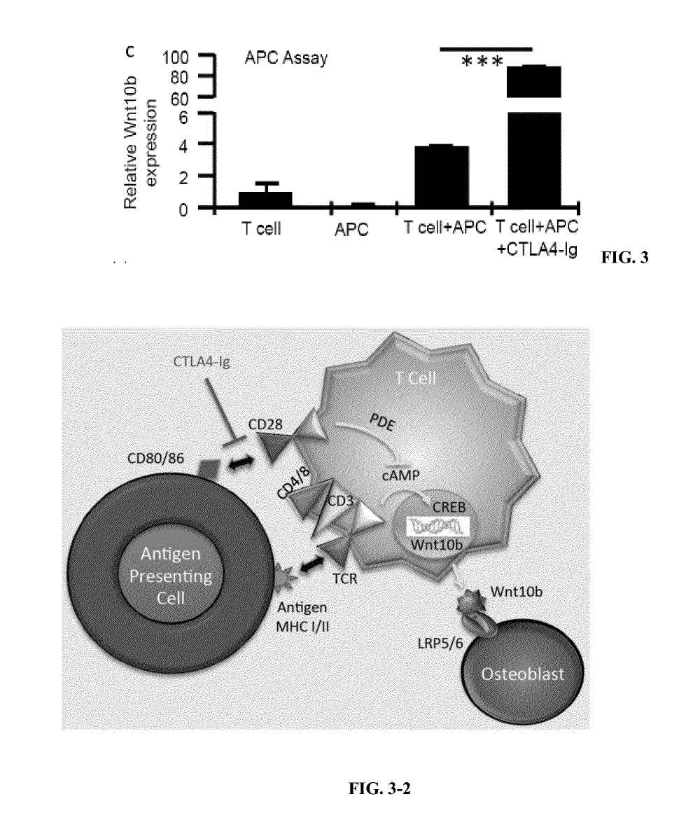

In the context of an impeded CD28 signal T-cell cAMP production would remain active leading to Wnt10b secretion and binding to Wnt receptors (LRPS and 6) on osteoblasts leading to their activation and new bone formation. This model is presented diagrammatically in FIG. 3D and is further supported by published gene array data demonstrating that WntlOb expression is upregulated in anergic T-cells.

The anabolic activity of intermittent parathyroid hormone (PTH) is mediated in part though T-cell production of Wnt10b, a Wnt ligand previously reported to be secreted by T-cells. Because PTH is a potent inducer of cAMP, it is believed that Wnt10b expression may be directly promoted by activation of cAMP in T-cells bypassing normal TCR mediated interactions with APC (TCR and CD28 signaling) leading to a potent sustained production of Wnt10b.

An interesting conundrum that is explained by this hypothesis is the question of why the genetic deletion of T-cells leads to an increase in bone resorption but fails to dramatically curtail bone formation while CD28 inhibition alone, induces bone formation. It is believed that WntlOb production is only elicited in contexts involving a significant impediment to CD28 signaling (such as exogenous application of CTLA4-Ig or intermittent administration of PTH). Consequently, under physiological conditions basal T-cell activation is relatively weak and hence little WntlOb is secreted. Even in inflammatory conditions CD28 activation during true APC-mediated T-cell activation would quickly silence Wnt10b expression.

CTLA4-Ig is protective of ovariectomy and continuous PTH-induced bone loss by blunting osteoclastic bone resorption driven by inflammatory cytokines such as RANKL and TNF secreted by T cells. Anabolic effects were not observed in these studies, and the anabolic effect of CTLA4-Ig is gradual but progressive, thus achieving significant bone gains over an extended period of time. Although, early changes in resorption may also occur following CTLA4-Ig administration, experiments suggest that the major net effect of chronic exposure, as used therapeutically in humans, is likely to be predominantly anabolic.

Consistent with high basal bone turnover in young mice rates of bone formation and bone accretion were robust in young mice treated with CTLA4-Ig. By contrast, bone accretion in skeletally mature mice was more modest. Femoral indices were not significantly increased, likely due to the rapid degradation of trabecular bone in the femurs of mice following peak BMD leaving a denuded template for osteoblasts to act on. Interestingly, although basal bone formation represented by serum osteocalcin was significantly diminished compared to younger mice, CTLA4-Ig did potently promote bone formation in these mice suggesting that an anabolic response was in fact underway and that a statistically significant response would be likely given additional time.

Administration of CTLA4-Ig to treat inflammatory diseases such as RA, may have multiple beneficial effects including reduced inflammation and reduced osteoclastic bone resorption driven by inflammation as well as due to direct inhibitory effects on osteoclasts. The present study further suggests bone anabolic activities due to T-cell release of Wnt10b may further promote a beneficial balance between bone formation and resorption.

Contrary to inflammatory contexts, experiments of basal bone turnover did not detect significant declines in CTx, an index of bone resorption, following chronic treatment with CTLA4-Ig. This was unexpected given that CTLA4-Ig is reported to mediate a direct anti-osteoclastic activity in vitro. A possible explanation is that early acute effects on osteoclasts are indeed observed early in the treatment but return to baseline over time.

CTLA4-Ig, a CD28 co-stimulation inhibitor, is a potent bone anabolic agent and promotes the production of Wnt10b by T-cells. Teriparatide, a fragment of PTH, administered intermittently is an FDA approved modality for stimulating bone accretion. CTLA4-Ig, e.g., Abatacept, ameliorates osteoporosis by stimulating bone formation, either as a stand-alone agent or in combination with anabolic or anti-catabolic agents.

### CTLA4-Ig Chimeras

In certain embodiment the disclosure relates to uses disclosed herein of CTLA4-Ig, i.e., a protein wherein one or more CTLA4 polypeptides are linked to an Fc region. The Fc portion may bind to one or more Fc receptors or Fc ligands. Fusion partners may be linked to any region of an Fc region, including at the N- or C-termini, or at some residue in-between the termini. CTLA4-Ig comprises a CTLA4 or a variant of CTLA4 as a fusion partner. The fusion of CTLA4 with an Ig Fc region is referred to herein as a CTLA4-Ig or CTLA4-Ig protein.

The CTLA4 polypeptide binds B7-1 and optionally B7-2. The target binding portion may comprise an amino acid sequence that is made up of all, any, or part of the human CTLA4 protein, e.g., all or part of (SEQ ID NO:1). M HVAQ PAVV LASS RGIA SFVC EYAS PGK(X¹) TEVR VTVL RQAD SQVTE VCAA (X²)Y(X³)M GNEL TF(X⁴)D DSIC TGTS SGNQ VNLT IQGL RAMD TGLY IC(X⁵)V ELMY PPPY YLGI GNGT QIYV IDPE PCPDS D wherein X¹ is Y, A, or H, X² is T or N, X³ is M or Y, X⁴ is L or E, and X⁵ is K or Q.

According to U.S. Patent Application Publication Number 2011/0287032, hereby incorporated by reference in its entirety, abatacept comprises a CTLA4-Ig polypeptide of the following sequence (SEQ ID NO:2) MHVAQ PAVVLASSRGIASFV CEYASPG KATEVRVTVLRQADSQV TEVCAATY MMGNELTFLDDSICTGTSSGNQVNL TIQGLRAMDTGLYICKVELM YPPPYYLGIGNGT QIYVIDPEPCPDS DQEPKSSDKTHT SPPSPAPELLGGS SVFLFPPKPKD TLMISRTPEVT CVVVDVSHEDPEVK FNWYVDGVEVHNA KTKPREEQYNSTYRWSVL TVLHQDWL NGKEYKCKVSNK ALPAPIEKTISKAKGQ PREPQVYTLPPSRDEL TKNQVSL TCLVKGF YPSDIAVEWESNGQPENNYKTTP PVLDSDGSFFL YSKL TVDKSRWQQGNVF SCSVMHEALHNHYTQKSLSLSPGK.

The CTLA4 variants optionally comprise at least one amino acid modification in a native CTLA4 protein or in SEQ ID NO:1. In this embodiment, one or more modifications are made at one or more of the following positions (numbering as in SEQ ID NO:2): 29, 30, 31, 33, 35, 49, 51, 53, 59, 61, 63, 64, 93, 95, 97, 98, 102, 103, 104, 105 or 106. In some embodiments, the modification is one or more of the following substitutions: A29E, A29F, A29H, A29K, A29N, A29Q, A29R, T30E, T3OH, T30R, T30V, E31D, E31I, E31M, E31T, E31V, R33E, R33F, R331, R33L, R33M, R33Q, R33T, R33W, R33Y, T35D, T35E, T35F, T35M, T35V, T35Y, A49D, A49E, A49F, A49T, A49W, A49Y, T51D, T51E, T51H, T51L, T51N, T51Q, T51R, T51S, T51V, M53E, M53F, M53H, M53Q, M53W, M53Y, T59H, T59I, T59L, T59N, T59Q, T59V, T59Y, L61A, L61D, L61E, L61F, L61G, L61H, L61I, L61,K, L61M, L61N, L61P, L61Q, L61R, L61S, L61T, L61V, L61W, L61Y, D63E, S64K, S64R, S64Y, K93D, K93E, K93F, K93H, K93N, K93Q, K93R, K93S, K93T, K93V, K93W, K93Y, E95D, E95H, E95L, E95Q, E95Y, M97D, M97F, M971, M97N, M97V, Y98F, Y98W, Y102F, Y102W, Y103D, Y103E, Y103F, Y103H, Y103N, Y103Q, Y103W, L104F, L104H, L104M, L104V, L104Y, G105D, G105E, I106E, and I106Y, Of particular use in some embodiments are CTLA4 variants that have one or more substitutions selected from A29H, T51N, M53Y, L61E, and K93Q, with combinations of particular use including A29H/K93Q, A29H/M53Y, A29H/T51N, T51N/K93Q, T51N/M53Y, A29H/L61E/K93Q, A29H/M53Y/K93Q, A29H/M53Y/L61E, A29H/T51N/L61E, M53Y/L61E/K93Q, T51N/L61E/K93Q, T51N/M53Y/L61E, A29H/M53Y/L61E/K93Q, A29H/T51N/L61E/K93Q, A29H/T51N/M53Y/K93Q, A29H/T51N/M53Y/L61E, T51N/M53Y/L61E/K93Q, and A29H/T51N/M53Y/L61E/K93Q.

As for all the lists of positions and substitutions herein, it should be understood that combinations of individual substitutions can be made, of any and all possible combinations, and that any individual position or substitution can be independently included or excluded from the list of possibilities. In general, as compared to the wild-type or parent CTLA4 (or Fc region), generally the variants of the invention have 1, 2, 3, 4, or 5 amino acid substitutions in the CTLA4 region, although in some cases more substitutions can be used, as long as the desired function is preserved. Similarly, as described below, the Fc domain may have substitutions in this manner as well. The CTLA4 variants generally preserve or enhance binding to one or more of the CTLA4 ligands, such as enhanced binding to B7-1 and/or B7-2.

The Fc portion are comprised of the Fc region or some portion of the Fc region of an antibody. Antibodies are immunoglobulins that bind a specific antigen. In most mammals, including humans and mice, antibodies are constructed from paired heavy and light polypeptide chains. The light and heavy chain variable regions show significant sequence diversity between antibodies, and are responsible for binding the target antigen. Each chain is made up of individual immunoglobulin (Ig) domains, and thus the generic term immunoglobulin is used for such proteins.

Traditional natural antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. IgA has several subclasses, including but not limited to IgA1 and IgA2. Thus, “isotype” as used herein is meant any of the classes and subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. The distinguishing features between these antibody classes are their constant regions, although subtler differences may exist in the variable region.

In certain embodiments, polypeptides are proteins that are fusions of CTLA4 with the Fc region of an antibody. By “Fc” or “Fc region”, as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. By “Fc polypeptide” as used herein is meant a polypeptide that comprises all or part of an Fc region. Fc polypeptides include antibodies, Fc fusions, isolated Fe's, and Fc fragments.

CTLA4 proteins may be linked to Fc regions via a linker. The term “linker” is used to denote polypeptides comprising two or more amino acid residues joined by peptide bonds and are used to link one or more antigen binding portions. A variety of linkers may find use in some embodiments described herein to covalently link Fc regions to a fusion partner. “Linker” herein is also referred to as “linker sequence”, “spacer”, “tethering sequence” or grammatical equivalents thereof. A number of strategies may be used to covalently link molecules together. These include, but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used. Useful linkers include glycine-serine polymers, including for example (GS)n, where n is an integer of at least one, glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use as linkers.

CTLA4-Ig proteins disclosed herein may comprise a variant CTLA4, a variant Fc region, or both a variant CTLA4 and a variant Fc region. A variant comprises one or more amino acid modifications relative to a parent CTLA4-Ig protein, wherein the amino acid modification(s) provide one or more described properties. By “amino acid” and “amino acid identity” as used herein is meant one of the 20 naturally occurring amino acids or any non-natural analogues that may be present at a specific, defined position. Thus “amino acid” as used herein means both naturally occurring and synthetic amino acids. For example, homophenylalanine, citrulline and noreleucine are considered. “Amino acid” also includes amino acid residues such as proline and hydroxyproline. The side chain may be in either the (R) or the (S) configuration. In the preferred embodiment, the amino acids are in the (S) or L-configuration. If non-naturally occurring side chains are used, non-amino acid substituents may be used, for example to prevent or retard in vivo degradation. An amino acid modification can be an amino acid substitution, insertion, and/or deletion in a polypeptide sequence. By “amino acid substitution” or “substitution” herein is meant the replacement of an amino acid at a particular position in a parent polypeptide sequence with another amino acid. By “amino acid insertion” or “insertion” as used herein is meant the addition of an amino acid at a particular position in a parent polypeptide sequence. By “amino acid deletion” or “deletion” as used herein is meant the removal of an amino acid at a particular position in a parent polypeptide sequence.

Antibody Fc regions contain carbohydrate at conserved positions in the constant regions of the heavy chain. Each antibody isotype has a distinct variety of N-linked carbohydrate structures. Aside from the carbohydrate attached to the heavy chain, up to 30% of human IgGs have a glycosylated Fab region. IgG has a single N-linked biantennary carbohydrate at Asn297 of the CH2 domain. For IgG from either serum or produced ex vivo in hybridomas or engineered cells, the IgG are heterogeneous with respect to the Asn297 linked carbohydrate. For human IgG, the core oligosaccharide normally consists of GlcNAc2Man3GlcNAc, with differing numbers of outer residues.

### Bone Conditions and Degenerative Diseases

In certain embodiments, the subject is diagnosed with, at risk of, or exhibiting symptoms of bone degenerative disease, osteoporosis, osteopenia, bone metastasis, multiple myeloma, osteogenesis imperfecta, hyperparathyroidism, or alcoholism. In certain embodiments, the subject is diagnosed with a bone density that is more than one or two standard deviations below the mean of a thirty year old subject of the same sex. In certain embodiments the subject is or is not diagnosed with rheumatoid arthritis.

Dual energy X-ray absorptiometry (DXA), quantitative computed tomography, and quantitative ultrasound are methods used to determine bone mineral density (BMD). In DXA two X-ray beams with different energy levels are aimed at bones. The bone mineral density can be determined from the absorption of each beam by bone after subtracting out soft tissue absorption. Osteoporosis is diagnosed when the bone mineral density is less than or equal to 2.5 standard deviations below that of a young adult reference population, e.g., 30 year old of the same sex. This is translated as a T-score. A T-score −1.0 or greater is considered “normal. A T-score between −1.0 and −2.5 is of low bone mass providing a diagnosis of osteopenia, and a T-score −2.5 or below is considered to be osteoporotic, providing a diagnosis of osteoporosis. When there has also been a low trauma-fracture, e.g., one that occurs as a result of a fall from a standing height, the subject is sometime referred to as having severe or established osteoporosis.

### Other Bone Growth Applications and Kits

In certain embodiments, the disclosure contemplates methods of improving bone growth by administering a therapeutic containing abatacept or belatacept or other molecule that binds with CD80, CD86, or CD28 providing inhibition of CD28 co-stimulation to a subject that has had a fracture, bone graft, or other surgery involving the skeleton or bone turnover. Methods contemplate oral administration, intravenous administration, subcutaneous, or direct injection at the desired site(s) of the subject.

Bone grafting is typically performed for spinal fusions, after cancerous bone removal, and in certain operations, e.g., plastic surgery. Synthetic bone grafts typically include a matrix that holds minerals and other salts. Natural bone has an intracellular matrix mainly composed of type I collagen, and some synthetic bone grafts include a collagen matrix. Synthetic bone grafts typically contain bone growth factors such as bone morphogenetic proteins (BMPs) because of their ability to induce bone growth in the matrix material. Recombinant human BMP-2 has been approved by the FDA in synthetic bone grafts such as INFUSE™. INFUSE™ is approved for open tibial shaft fractures, lumbar interbody fusion, and sinus and alveolar ridge augmentations.

In certain embodiments, the disclosure contemplates administration of systemic abatacept or belatacept in conjunction with a bone graft with or without a bone morphogenetic protein and/or another growth factor. In certain embodiments, bone graft compositions comprise abatacept or belatacept or other molecule that binds with CD80, CD86, or CD28 providing inhibition of CD28 co-stimulation, and a bone morphogenetic protein and/or another growth factor. Typically, the bone morphogenetic protein is BMP-2, BMP-5, or BMP-7. In certain embodiments, the graft composition comprises calcium phosphates and/or bone granules, hydroxyapatite and/or beta-tricalcium phosphate, alpha-tricalcium phosphate, polysaccharides or combinations thereof. Crushed bone granules, typically obtained from the subject, are optionally added to the graft composition.

Bone grafting is possible because bone tissue, unlike most other tissues, has the ability to regenerate if provided the space into which to grow with appropriate chemical signals. With regard to synthetic grafts, as native bone grows, it typically replaces most or all of the artificial graft material, resulting in an integrated region of new bone. However, with regard to certain embodiments of the disclosure, it is not intended that new bone must remove all artificial material. In addition, with regard to certain embodiments of the disclosure, it is not intended that graft location need contact any other bone of the skeletal system.

In certain embodiments, the disclosure relates to a method of forming bone comprising implanting a graft composition comprising abatacept or belatacept or other molecule that binds with CD80, CD86, or CD28 providing inhibition of CD28 co-stimulation, in a subject optionally in conjunction with systemic abatacept or belatacept administration. In certain embodiments, the disclosure relates to methods of forming bone comprising implanting a graft composition comprising a bone morphogenetic protein and abatacept or belatacept or other molecule that binds with CD80, CD86, or CD28 providing inhibition of CD28 co-stimulation, in a subject. The graft may be the result of a void created by surgical removal or created as a result of an attempt to correct a physical abnormality of a bone, such as but not limited to, cranial bones; frontal, parietal, temporal, occipital, sphenoid, ethmoid; facial bones; mandible, maxilla, palatine, zygomatic, nasal, lacrimal, vomer, inferior nasal conchae; shoulder girdle; scapula or shoulder blade, clavicle or collarbone; in the thorax; sternum, manubrium, gladiolus, and xiphoid process, ribs; in the vertebral column; cervical vertebrae, thoracic vertebrae; lumbar vertebrae; in the arms, humerus, radius, ulna; in the pelvis; coccyx; sacrum, hip bone (innominate bone or coxal bone); in the legs; femur, patella, tibia, and fibula. It is contemplated that the graft may be added for cosmetic purposes, e.g., cheek augmentation. In the case of a broken bone or removal of a bone during surgery, it may be desirable to secure movement of bone structure with a fixation system and remove the system after bone forms in the implanted graft.

With regard to prostheses, it may be desirable to grow bone between existing bone and an implanted device, or in preparation of an implanted device, such as in the case of a hip replacement, knee replacement, and dental implant, i.e., artificial tooth root used to support restorations that resemble a tooth or group of teeth.

In some embodiments, the disclosure relates to three-dimensional structures made of biocompatible and biodegradable bone graft materials in the shape of the bone infused with compositions disclosed herein to promote bone growth. Implants can be used to support a number of prostheses. A typical implant consists of a titanium device. In certain embodiments, the graft compositions disclosed herein contain implants.

With regard to a sinus augmentation or alveolar ridge augmentation, surgery may be performed as an outpatient under general anesthesia, oral conscious sedation, nitrous oxide sedation, intravenous sedation or under local anesthesia. Bone grafting is used in cases where there is a lack of adequate maxillary or mandibular bone in terms of depth or thickness. Sufficient bone is needed in three dimensions to securely integrate with the root-like implant. Improved bone height is important to assure ample anchorage of the root-like shape of the implant.

In a typical procedure, the clinician creates a large flap of the gingiva or gum to fully expose the bone at the graft site, performs one or several types of block and on-lay grafts in and on existing bone, then installs a membrane designed to repel unwanted infection-causing bacteria. Then the mucosa is carefully sutured over the site. Together with a course of systemic antibiotics and topical antibacterial mouth rinses, the graft site is allowed to heal. The bone graft produces live vascular bone and is therefore suitable as a foundation for the dental implants.

In certain embodiments, the disclosure relates to methods of performing spinal fusion using compositions disclosed herein. Typically this procedure is used to eliminate the pain caused by abnormal motion of the vertebrae by immobilizing the vertebrae themselves. Spinal fusion is often done in the lumbar region of the spine, but the term is not intended to be limited to method of fusing lumbar vertebrae. Patients desiring spinal fusion may have neurological deficits or severe pain which has not responded to conservative treatment. Conditions where spinal fusion may be considered include, but are not limited to, degenerative disc disease, spinal disc herniation, discogenic pain, spinal tumor, vertebral fracture, scoliosis, kyphosis (i.e, Scheuermann's disease), spondylolisthesis, or spondylosis.

In certain embodiments, different methods of lumbar spinal fusion may be used in conjunction with each other. In one method, one places the bone graft between the transverse processes in the back of the spine. These vertebrae are fixed in place with screws and/or wire through the pedicles of each vertebra attaching to a metal rod on each side of the vertebrae. In another method, one places the bone graft between the vertebra in the area usually occupied by the intervertebral disc. In preparation for the spinal fusion, the disc is removed entirely. A device may be placed between the vertebrae to maintain spine alignment and disc height. The intervertebral device may be made from either plastic or titanium or other suitable material. The fusion then occurs between the endplates of the vertebrae. Using both types of fusion are contemplated.

In certain embodiments, the disclosure relates to methods of growing bone at a desired area by locally administering a pharmaceutical formulation containing abatacept or belatacept or other molecule that binds with CD80, CD86, or CD28 providing inhibition of CD28 co-stimulation, to the area of desired bone growth with or without the presence of a graft composition or bone growth matrix optionally in combination with a growth factor such as a bone morphogenetic protein. In certain embodiments, the disclosure contemplates administering the pharmaceutical compositions between vertebra, e.g., in the area usually occupied by the intervertebral disc, to form a spinal fusion.

In some embodiments, the disclosure relates to kits comprising a graft composition, abatacept or belatacept or other molecule that binds with CD80, CD86, or CD28 providing inhibition of CD28 co-stimulation, and a graft matrix. In certain embodiments, the kits further comprise a bone morphogenetic protein and/or another growth factor. In certain embodiments, the kits further comprise a transfer device, such as a syringe or pipette.

### Antibodies, Fragments, Chimera, Antibody Mimetics, and Aptamers

In certain embodiments, the disclosure contemplates targeting moieties in any of the disclosed embodiments that are antibodies or fragments or chimera, antibody mimetics, or aptamers or other molecule that binds with CD80, CD86, or CD28 providing inhibition of CD28 co-stimulation.

Numerous methods known to those skilled in the art are available for obtaining antibodies or antigen-binding fragments thereof For example, antibodies can be produced using recombinant DNA methods (U.S. Pat. No. 4,816,567). Monoclonal antibodies may also be produced by generation of hybridomas in accordance with known methods. Hybridomas formed in this manner are then screened using standard methods, such as enzyme-linked immunosorbent assay (ELISA) and surface plasmon resonance analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

The modular structure of antibodies makes it possible to remove constant domains in order to reduce size and still retain antigen binding specificity. Engineered antibody fragments allow one to create antibody libraries. A single-chain antibody (scFv) is an antibody fragment where the variable domains of the heavy (V_{H}) and light chains (V_{L}) are combined with a flexible polypeptide linker. The scFv and Fab fragments are both monovalent binders but they can be engineered into multivalent binders to gain avidity effects. One exemplary method of making antibodies and fragments includes screening protein expression libraries, e.g., phage or ribosome display libraries. Phage display is described, for example, in U.S. Pat. No. 5,223,409.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. U.S. Pat. No. 7,064,244.

Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. An antibody or fragment thereof may also be modified by specific deletion of human T cell epitopes or “deimmunization” by the methods disclosed in U.S. Pat. No. 7,125,689 and U.S. Pat. No. 7,264,806. Briefly, the heavy and light chain variable domains of an antibody can be analyzed for peptides that bind to MHC Class II; these peptides represent potential T-cell epitopes. For detection of potential T-cell epitopes, a computer modeling approach termed “peptide threading” can be applied, and in addition a database of human MHC class II binding peptides can be searched for motifs present in the VH and VL sequences. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. The V BASE directory provides a comprehensive directory of human immunoglobulin variable region sequences. These sequences can be used as a source of human sequence, e.g., for framework regions and CDRs. Consensus human framework regions can also be used, e.g., as described in U.S. Pat. No. 6,300,064.

Antibody mimetics or engineered affinity proteins are polypeptide based targeting moieties that can specifically bind to targets but are not specifically derived from antibody V_{H} and V_{L} sequences. Typically, a protein motif is recognized to be conserved among a number of proteins. One can artificially create libraries of these polypeptides with amino acid diversity and screen them for binding to targets through phage, yeast, bacterial display systems, cell-free selections, and non-display systems. See Gronwall & Stahl, J Biotechnology, 2009, 140(3-4), 254-269, hereby incorporated by reference in its entirety. Antibody mimetics include affibody molecules, affilins, affitins, anticalins, avimers, darpins, fynomers, kunitz domain peptides, and monobodies.

Affibody molecules are based on a protein domain derived from staphylococcal protein A (SPA). SPA protein domain denoted Z consists of three α-helices forming a bundle structure and binds the Fc protion of human IgG1. A combinatorial library may be created by varying surface exposed residues involved in the native interaction with Fc. Affinity proteins can be isolated from the library by phage display selection technology.

Monobodies, sometimes referred to as adnectins, are antibody mimics based on the scaffold of the fibronectin type III domain (FN3). See Koide et al., Methods Mol. Biol. 2007, 352: 95-109, hereby incorporated by reference in its entirety. FN3 is a 10 kDa, β-sheet domain, that resembles the V_{H} domain of an antibody with three distinct CDR-like loops, but lack disulfide bonds. FN3 libraries with randomized loops have successfully generated binders via phage display (M13 gene 3, gene 8; T7), mRNA display, yeast display and yeast two-hybrid systems. See Bloom & Calabro, Drug Discovery Today, 2009, 14(19-20):949-955, hereby incorporated by reference in its entirety.

Anticalins, sometimes referred to as lipocalins, are a group of proteins characterized by a structurally conserved rigid β-barrel structure and four flexible loops. The variable loop structures form an entry to a ligand-binding cavity. Several libraries have been constructed based on natural human lipocalins, i.e., ApoD, NGAL, and Tlc. Anticalins have been generated for targeting the cytotoxic T-lymphocyte antigen-4 (CTLA-4). See Skerra, FEBS J., 275 (2008), pp. 2677-2683, and Binder et al., J Mol Biol., 2010, 400(4):783-802., both hereby incorporated by reference in their entirety.

The ankyrin repeat (AR) protein is composed repeat domains consisting of a β-turn followed by two α-helices. Natural ankyrin repeat proteins normally consist of four to six repeats. The ankyrin repeats form a basis for darpins (designed ankyrin repeat protein) which is a scaffold comprised of repeats of an artificial consensus ankyrin repeat domain. Combinatorial libraries have been created by randomizing residues in one repeat domain. Different numbers of the generated repeat modules can be connected together and flanked on each side by a capping repeat. The darpin libraries are typically denoted NxC, where N stands for the N-terminal capping unit, C stands for the C-terminal capping domain and x for the number of library repeat domains, typically between two to four. See Zahnd et al., J. Mol. Biol., 2007, 369:1015-1028, hereby incorporated by reference in its entirety.

Aptamers refer to affinity binding molecules identified from random proteins or nucleic acids libraries. Peptide aptamers have been selected from random loop libraries displayed on TrxA. See Borghouts et al., Expert Opin. Biol. Ther., 2005, 5:783-797, hereby incorporated by reference in its entirety. SELEX (“Systematic Evolution of Ligands by Exponential Enrichment”) is a combinatorial chemistry technique for producing oligonucleotides of either single-stranded DNA or RNA that specifically bind to a target. Standard details on generating nucleic acid aptamers can be found in U.S. Pat. No. 5,475,096, and U.S. Pat. No. 5,270,163. The SELEX process provides a class of products which are referred to as nucleic acid ligands or aptamers, which has the property of binding specifically to a desired target compound or molecule. Each SELEX-identified nucleic acid ligand is a specific ligand of a given target compound or molecule. The SELEX process is based on the fact that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

### EXAMPLES

Treatment of Mice with CTLA4-Ig Leads to an Elevation in BMD, and Enhanced Trabecular and Cortical Bone Volume.

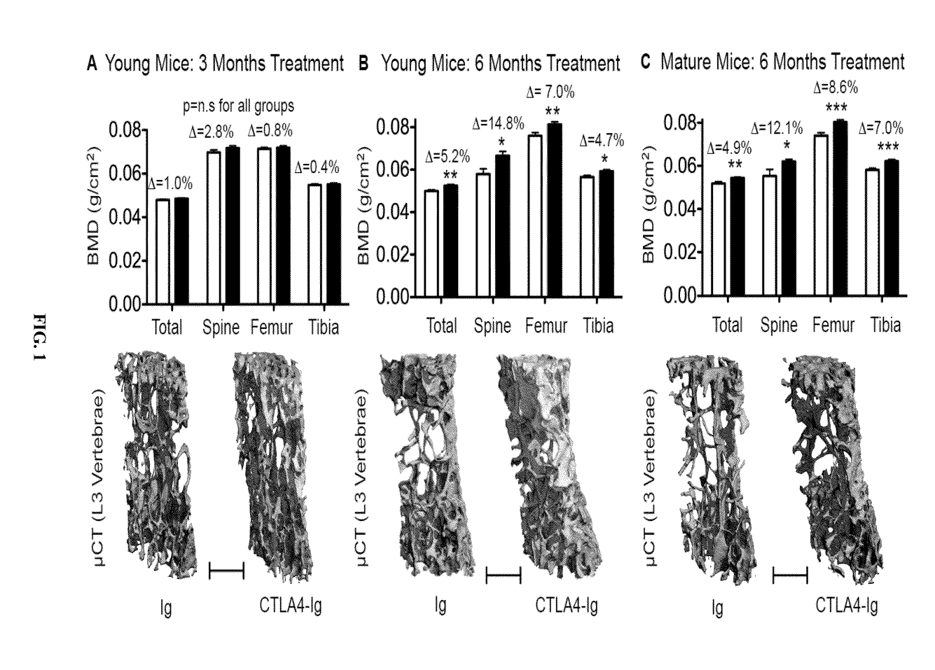

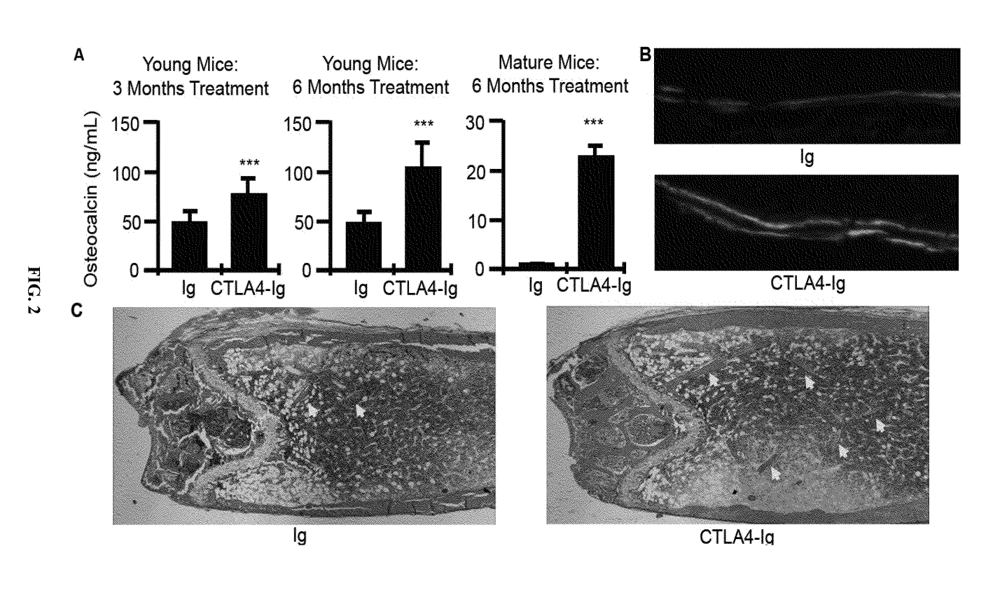

In order to investigate the net effect of the immunosuppressant agent CTLA4-Ig on physiological basal bone modeling, CTLA4-Ig or irrelevant isotype control Ig was injected into young female (6-week-old) C57BL6 mice. BMD was examined after 12 and 26 weeks by bone densitometry using DEXA. Although CTLA4-Ig administration failed to cause any significant change in BMD compared to control immunoglobulin (Ig) control at 3 months (FIG. 1A), by 6 months of treatment CTLA4-Ig injected mice displayed a significant increase in total body BMD and increases at specific anatomical sites including femur and tibia (left and right femur or tibia for each mouse independently averaged) and lumbar spine (FIG. 1B).

To assess the effect of chronic CTLA4-Ig treatment on the remodeling skeletons of adult mice, 5-month-old mice were treated with CTLA4-Ig or control (Ig) for six months. As with young mice total body BMD was significantly elevated as was BMD at femur, lumbar spine and tibia (FIG. 1C).

To provide independent evaluations of cortical and cancellous bone, μCT of lumbar vertebrae and femurs were employed. Representative μCT reconstructions of vertebral trabecular bone are shown for young skeletally immature mice (6 weeks of age) treated for 3 months (FIG. 1A, lower panel), young mice treated for 6 months (FIG. 1B, lower panel) and in skeletally mature (5 months of age) mice treated for 6 months (FIG. 1C, lower panel).

Quantitative microarchitectural indices of trabecular bone structure were further computed for young mice receiving 3 and 6 months of CTLA4-Ig treatment and for mature mice receiving 6 months of treatment. Vertebral trabecular bone volume fraction (BV/TV) was significantly increased in young mice receiving CTLA4-Ig for both 3 and 6 months consistent with a decline in trabecular separation (Tb. Sp.), reflecting the amount of bone free space. Trabecular thickness (Tb. Th.) and trabecular number (Tb. N.) were significantly increased in young mice by 6 months of treatment but fell just short of significance at 3 months, suggesting a slow accumulation of bone volume over time. Bone accretion in mature mice was slower than in younger animals and BV/TV fell just short of statistical significance although Tb. N. and Tb. Sp., were significantly different to Ig treated controls.

Femoral trabecular BV/TV was significantly increased at both 3 and 6 months of treatment in young animals although changes in Tb. Sp., Tb. Th., and Tb. N. did not achieve statistical significance. In both cases Tb. Th., showed the largest increases and just narrowly fell short of significance at 6 months of treatment. These data suggest that CTLA4-Ig may work predominantly by expanding the thickness of preexisting trabecular spicules rather than catalyzing de novo synthesis of new template.

Cortical bone was quantified in the mid-femoral diaphysis and revealed a significant increase in cortical thickness (Co. Th.) in young mice by 6 months while Cortical area (Co. Ar.) was increased in magnitude by 6 months but fell just short of statistical significance. Neither index reached statistical significance in mature mice. Because total body and all anatomical analyses of BMD by DEXA (FIG. 1A) revealed significant increases in BMD in mature animals the data suggest that small increases in cortical bone across large areas may account for the increases resolved by DEXA but not by μCT.

### CTLA4-Ig Enhances Biochemical Indices of Bone Formation but not Indices of Bone Resorption In Vivo.

To assess the rates of bone resorption and bone formation in vivo bone turnover markers were quantified in the serum of CTLA4-Ig and Ig treated mice. Serum C-terminal telopeptide of collagen (CTx), a sensitive and specific index of bone resorption was not significantly changed between Ig and CTLA4-Ig treated groups in either young or mature mice and irrespective of time on treatment (CTx: Young mice 6 months (Ig: 24.7±5.7 vs. CTLA4-Ig; 23.8±9.4) and mature mice 6 months (Ig: 13.2±5.9 vs. CTLA4-Ig 14.2±3.3). Mean±SD, P=N.S.

Consistent with these data RT-PCR of total bone marrow revealed no significant alterations in expression of OPG and RANKL at 26 weeks (2-AACT of OPG: Ig (1.00±0.18) vs. CTLA4-Ig (0.87±0.17), and 2-AACT of RANKL: Ig (1.00±0.04) vs. CTLA4-Ig (0.88±0.05); mean±SD, P=NS).

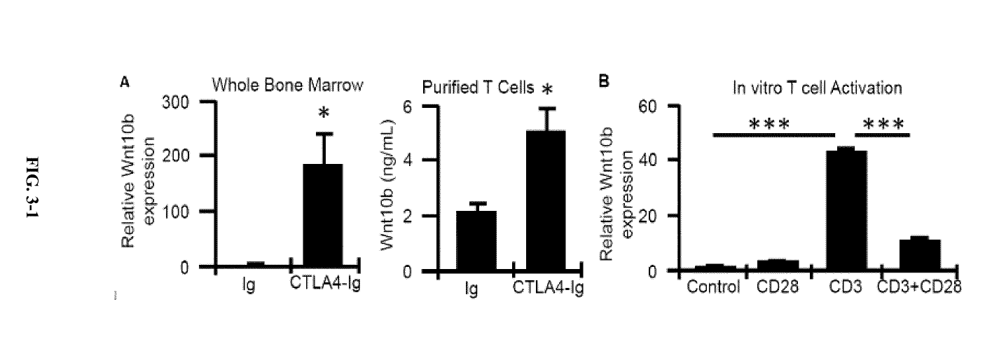

By contrast, serum osteocalcin, a biochemical marker of in vivo bone formation was dramatically increased in young mice treated with CTLA4-Ig for 3 months, 6 months and in mature mice treated for 6 months (FIG. 2A). These data suggest that bone accretion was a consequence of elevated bone formation.

### CTLA4-Ig Enhances Indices of Bone Formation Quantified by Bone Histomorphometry.

As osteocalcin reflects global bone formation across all bone surfaces we further employed quantitative bone histomorphometry to access bone formation in young mice treated for 6 months with CTLA4-Ig at a tissue and cellular level. Three indices of bone formation mineralizing surface (MS), mineral apposition rate (MAR) and bone formation rate (BFR) normalized for TV (BFR/TV) were all significantly increased. When BFR was normalized for bone surface (BS) it fell just short of significance while BFR normalized for BV was unchanged from control. The static indices, osteoblast surface (ObS)/BS and number of osteoblasts (N.Ob)/BS, showed a non-significant decline indicating that the long term effect of CTLA4-Ig was not to increase osteoblast number, but rather induce activation of pre-existing osteoblasts driving a wave of new bone formation. As a consequence, the number of osteoblasts and the area of bone covered by osteoblasts both appeared to decline as a result of the significantly increased bone surfaces. BFR/TV, which normalizes for total bone area is the index that correlates most closely with bone turnover markers such as osteocalcin. Similarly, small non-significant declines in osteoclast number (N.Oc/BS) and surface (OcS/BS) were observed, and likely also reflect the relative increase in BS, rather than long term direct effect of CTLA4-Ig on osteoclast number. Cancellous structural indices including BV, BV/TV, Tb. N. and Tb. Sp. as computed by histomorphometry all showed robust changes supporting gain of bone mass as observed in the μCT data. Surprisingly, the BV/TV of femoral bone determined by histomorphometry was more than twice that observed by μCT. In principle μCT is a more robust and reliable measure of bone volume and structure than histomorphometry as μCT reflects a true 3D quantification of a relatively large segment of bone while histomorphometry is a 2D representation of a comparatively small number of slices. This diminished precision likely accounts for the apparent discrepancy.

Representative double calcein labels from which MAR and BFR indices were calculated are shown in FIG. 2B and reveal enhanced bone formation in young CTLA4-Ig treated animals at 6 months of treatment. Representative photomicroscopy images of Goldner Trichrome stained femoral sections are shown in FIG. 2C and show enhanced numbers of trabecular elements in the femoral metaphysis (yellow arrows) and increased bone thickness in the epiphyses above the growth plate (red arrows).

The Anabolic Wnt Ligand, Wnt10b is Significantly Elevated in Total Bone Marrow and Purified T-Cells from CTLA4-Ig Treated Mice.

T-cells have the capacity to secrete Wnt10b, a potent bone anabolic Wnt ligand. As a possible explanation for the anabolic activity of CTLA4-Ig we quantified WntlOb expression by CTLA4-Ig and Ig treated mice in the bone marrow using real time RT-PCR and in conditioned medium from purified CD3+ T-cells by ELISA (FIG. 3A). Wnt10b expression was found to be significantly elevated in the bone marrow of CTLA4-Ig treated animals and protein production by purified CD3+ T-cells, suggesting involvement of Wnt10b by T-cells in the anabolic activity of CTLA4-Ig.

### CD28 Inhibits Wnt10b Expression by Activated T-Cells In Vitro, While CTLA4-Ig-Supression of CD28 Signaling Amplifies Wnt10b Expression Induced by Antigen Presentation In Vitro.

To further explore the mechanism of T-cell WntlOb production by CTLA4-Ig, T-cells we purified and activated in vitro using CD3e activating antibody in the presence or absence of activating CD28 antibody. Activation of CD3 led to a significant upregulation of Wnt10b expression at 24 hr (FIG. 3B). Activation of CD28 alone had no significant effect on Wnt10b but potently inhibited WntlOb expression induced by CD3. CTLA4-Ig may thus promote Wnt10b expression in T-cells by blocking the interaction of CD80/CD86 on APC with T-cell expressed CD28, a negative signal for Wnt10b expression. To test this hypothesis directly an in vitro APC assay was performed in which purified dendritic cells were used as APC to express Ova antigen to CD8+ T-cells expressing a monoclonal TCR with Ova-specific recognition. Presentation of Ova by APC to T cells led to induction of Wnt10b expression that was potently super-induced by addition of CTLA4-Ig to the culture (FIG. 3C).

### Methods

Mice were housed under specific pathogen free conditions and were fed gamma-irradiated 5V02 mouse chow (Purina Mills, St. Louis, Mo.), and autoclaved water ad libitum. The animal facility was kept at 23±1° C., with 50% relative humidity and a 12/12 light/dark cycle. Young (6 weeks of age) female C57BL6 WT and OTI mice were from Jackson Labs (Bar Harbor, Me.) and skeletally mature (5 month old) mice were from the National Institute on Aging (NIA) aged mouse colony at Charles River Laboratories (Wilmington, Mass.). WT mice were injected with 10 mg/Kg CTLA4-Ig (Orencia: Bristol-Myers Squibb) twice weekly intraperitoneally or with human IgG (Lampire Biological Laboratories, Pipersville, Pa.) for 3 or 6 months (26 weeks) as indicated. Skeletally mature mice comprised 8 Ig and 7 CTLA4-Ig mice/group while young mice treated for 3 months comprised 10 mice/group. Young mice treated for 6 months comprised 12 mice/group however, one extreme outlier in the CTLA4-Ig group with a final mean BV/TV of 0.12, falling 3 SD below the average and well below even the WT mean value of 2.07, was eliminated from the μCT and histomorphometry analysis. One bone used for histomorphometry was damaged during processing and had no quantifiable bone or cells.

Bone densitometry. BMD (g/cm2) quantifications were performed in anesthetized mice by DEXA using a PIXImus 2 bone densitometer (GE Medical Systems). Total body DEXA was performed and region of interest boxes placed to quantify anatomical sites including lumbar spine, femur and tibia. The left and right femurs and left and right tibias were averaged for each mouse and the mean used for group calculations.

Micro-Computed Tomography. μCT was performed in L3 vertebrae and femoral metaphysis ex vivo to assess trabecular bone microarchitecture using a μCT40 scanner (Scanco Medical AG, Bruettisellen, Switzerland) calibrated weekly with a factory-supplied phantom. A total of 405 tomographic slices were taken at the L3 vertebra (total area of 2.4 mm) and 100 tomographic slices at the distal femoral metaphysis and trabecular bone segmented from the cortical shell for a total area of 0.6 mm beginning approximately 0.5 mm from the distal growth plate. Projection images were reconstructed using the auto-contour function for trabecular bone. Cortical bone was quantified at the femoral mid-diaphysis from 100 tomographic slices. Representative vertebral samples based on mean BV/TV were reconstructed in 3D to generate visual representations. Indices and units were standardized.

Quantitative Bone Histomorphometry. Bone histomorphometry was performed on trichrome-stained plastic-embedded sections of calcein labeled femurs from Ig and CTLA4-Ig injected mice.

Biochemical indices of bone turnover. CTx, and osteocalcin were quantified in mice serum using RATlaps (CTx) and Rat-MID (osteocalcin) ELISAs (Immunodiagnostic Systems Inc. Fountain Hills Ariz.).

Wnt10b ELISA. WntlOb protein was determined in 24 hr conditioned media from negatively immunomagnetically purified CD3 T-cells (Miltenyi Biotech. Auburn, Calif.) using a WntlOb ELISA (USCN Life Science Inc., Wuhan, P.R. China).

Real-time RT-PCR. Total RNA was extracted from whole nucleated bone marrow, flushed from long bones and dissolved in Trizol Reagent. Real-time RT-PCR was performed on an ABI Prism 7000 instrument (Applied Biosystems, Foster City, Calif.) as previously described (10) using commercial (Applied Biosystems) master mix and primer sets and probes for murine Wnt10b (Mm00442104), OPG (Mm 001205928), RANKL (Mm 00441906) and β-actin (Mm 00607939). Changes were calculated using the 2-ΔΔCt method with normalization to β-actin.

T cell activation assays. Plates were coated overnight with activating anti-mouse CD3e (5 ug/ml) and/or anti-mouse CD28 (25 ug/ml) antibodies in sterile PBS (eBioscience, San Diego Calif.). Splenic T-cells were isolated using a CD3 Pan T cell isolation kit (Miltenyi Biotec.) and 12 replicate wells plated at 2×10⁷ cells/well in 24 well plates in 750 μl of RPMI1640+5% FBS for 24 hours and then dissolved in Trizol for RNA isolation and real time RT-PCR for WntlOb expression as described above.

APC Assays: Immunomagnetically purified (Miltenyi Biotech.) splenic CD11c dendritic cells were used as APC and plated in triplicate at 150,000 cells/well in complete RPMI1640+10% FBS and incubated for 4 h at 37° C. with 1 μM antigen (ovalbumin (Ova) peptide, (SIINFEKL) from Invivogen, San Diego, Calif.) followed by two washes in medium. CD8+ T-cells expressing a monoclonal Ova specific transgenic TCR were purified from spleens of OTI mice and 1 million T-cells incubated with Ova presenting APC±CTLA4-Ig for 24 hr. Non-adherent T-cells and adherent dendritic cells were separated and dissolved in Trizol for RNA isolation and real time RT-PCR for Wnt10b expression.

1. A method of growing bone or increasing bone mineral density and/or volume comprising administering an effective amount of a molecule that binds with CD80, CD86, or CD28 providing inhibition of CD28 co-stimulation to a subject in need thereof.

2. The method of claim 1, wherein the molecule is selected from antibodies, antibody fragments, antibody chimera, antibody mimetics, and aptamers.

3. The method of claim 1, wherein molecule comprises a polypeptide comprising 100 or more amino acids that overlap with a human CTLA-4 (Cytotoxic T-Lymphocyte Antigen 4) extra cellular domain sequence.

4. The method of claim 3, wherein the polypeptide comprises SEQ ID NO:1 M HVAQ PAVV LASS RGIA SFVC EYAS PGK(X¹) TEVR VTVL RQAD SQVTE VCAA (X²)Y(X³)M GNEL TF(X⁴)D DSIC TGTS SGNQ VNLT IQGL RAMD TGLY IC(X⁵)V ELMY PPPY YLGI GNGT QIYV IDPE PCPDS D wherein X¹ is Y, A, or H, X² is T or N, X³ is M or Y, X⁴ is L or E, and X⁵ is K or Q.

5. The method of claim 3, wherein the polypeptide comprises a human immunoglobulin.

6. The method of claim 3, wherein the polypeptide is abatacept or belatacept.

7. The method of claim 1, wherein the subject is diagnosed with, at risk of, or exhibiting symptoms of bone degenerative disease, osteoporosis, osteopenia, osteitis deformans, bone metastasis, multiple myeloma, osteogenesis imperfect, hyperparathyroidism, alcoholism, or HIV.

8. The method of claim 1, wherein the subject is diagnosed with a bone density that is more than one or two standard deviations below the mean of a thirty year old subject of the same sex.

9. The method of claim 1, wherein the subject is premenopausal woman, male under the age of 50, male or female under the age of 40 or 30.

10. The method of claim 1, wherein the subject is taking or has taken a glucocorticoid therapy.

11. The method of claim 1, wherein the polypeptide is administered in combination with teriparatide, anabolic parathyroid hormone or fragment, calcium, vitamin D, strontium ranelate, raloxifene, denosumab, estrogen, testosterone, bisphosphonates such as pamidronate, alendronate, risedronate, medronate, oxidronate, etidronate, clodronate, tiudronate, neridronate, olpadronate, ibandronate, orzoledronate, or statins such as atorvastatin, cerivastatin, fluvastatin, lovastatin, mevastatin, pitavastatin, pravastatin, rosuvastatin, or simavastatin, or a glucocorticoid such as hydrocortisone, cortisone, prednisone, prednisolone, methylprednisolone, dexamethasone, betamethasone, fludrocortisone, deoxycorticosterone, or aldosterone.

12. The method of claim 1, wherein the subject is or is not diagnosed with rheumatoid arthritis.

13. The method of claim 1, wherein the subject is diagnosed with a bone fracture or received spinal fusion surgery.

14. An osteogenic promoting implant comprising a molecule of claim 1.

15. The implant of claim 14, further comprising a bone morphogenetic protein selected from BMP-2, BMP-5, or BMP-7.

16. The implant of claim 14, further comprising calcium phosphates and/or bone granules, hydroxyapatite and/or beta-tricalcium phosphate, alpha-tricalcium phosphate, polysaccharides or combinations thereof.

17. The implant of claim 14 further comprising crushed bone granules.

18. The bone graft composition of claim 14 further comprising a hydrogel matrix or collagen matrix.

19. A method of growing bone comprising inserting a bone graft composition in an area of a subject of desired bone growth and administering an effective amount of a molecule that binds with CD80, CD86, or CD28 to the subject.

20. The method of claim 19, wherein the molecule is abatacept or belatacept.


SEQUENCE LISTING (2 sequences)

SEQ ID NO: 1 - protein, 124 aa, Homo sapiens
  MISC_FEATURE (29)..(29): Where Xaa is Alanine or Tryosine or Histidine
MHVAQPAVVLASSRGIASFVCEYASPGKXTEVRVTVLRQADSQVTEVCAAXYXMGNELTFXDDSICTGTSSGNQVNLTIQGLRAMDTGLYICXVELMYPPPYYLGIGNGTQIYVIDPEPCPDSD

SEQ ID NO: 2 - protein, 356 aa, Homo sapiens
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK